(19) United States
(12) Patent Application Publication
(10) Pub. No.: US 2019/0105648 A1
(43) Pub. Date: Apr. 11, 2019

(54) CATALYST BASED ON CATECHOLAMINE AND ITS USE IN A HYDROTREATMENT AND/OR HYDROCRACKING PROCESS

(71) Applicant: IFP Energies nouvelles, Rueil-Malmaison Cedex (FR)

(72) Inventors: Dorothee LAURENTI, Neyron (FR); Rajesh MUNIRATHINAM, Lyon (FR); Gerhard PIRNGRUBER, Charly (FR); Denis UZIO, Oullins (FR)

(73) Assignee: IFP Energies nouvelles, Rueil-Malmaison Cedex (FR)

(21) Appl. No.: 16/089,250

(22) PCT Filed: Mar. 1, 2017
(86) PCT No.: PCT/EP2017/054687

§ 371 Date: Sep. 27, 2018

(30) Foreign Application Priority Data
Mar. 30, 2016 (FR) 1652743

(51) Int. Cl. B01J 31/34; B01J 27/19; B01J 31/02; B01J 21/04; B01J 21/08; C07C 5/10; C10G 45/08

(52) U.S. Cl. CPC B01J 31/34; C07C 2527/186; B01J 31/0271; B01J 21/04; B01J 21/08; C07C 5/10; C10G 45/08; B01J 2531/002; B01J 2231/646; C07C 2523/75; C07C 2531/02; C07C 2521/04; C07C 2521/08; C10G 2300/104; C10G 2300/207; C10G 2400/02; C07C 2523/84; B01J 27/19

(57) ABSTRACT

The invention concerns a catalyst comprising a support based on alumina or silica or silica-alumina, at least one element selected from group VIII and/or group VIB, and at least one catecholamine. The invention also concerns the process for the preparation of said catalyst and its use in a hydrotreatment and/or hydrocracking process.

The invention relates to a catalyst containing a catecholamine, to its preparation method and to its use in the field of hydrotreatment and/or hydrocracking.

A catalyst for the hydrotreatment of hydrocarbon cuts is intended to eliminate sulphur-containing or nitrogen-containing compounds contained therein in order to comply with current specifications (sulphur content, aromatics content, etc.) for a given application (automobile fuel, gasoline or gas oil, domestic fuel, jet fuel). It may also be used to pre-treat a this feed in order in order to eliminate impurities therefrom or to hydrogenate it before it undergoes the various transformation processes such as, for example, reforming processes, vacuum distillate hydrocracking processes, catalytic cracking, or hydroconversion of atmospheric or vacuum residues. The composition and use of hydrotreatment catalysts have been described particularly well in the article by B. S. Clausen, H. T. Topsøe and F. E. Massoth, published in Catalysis Science and Technology, volume 11 (1996), Springer-Verlag.

Tightening of fuel standards in the European Community (Journal Officiel de L'Union européenne [Official Journal of the European Union] L76, 22 Mar. 2003, Directive 2003/70/CE, pages L76/10-L76/19) has constrained refiners to reduce to a very substantial extent the sulphur content in diesel fuels and gasolines (to a maximum of 10 parts per million by weight (ppm) of sulphur on 1 Jan. 2009, in contrast to 50 ppm on 1 Jan. 2005). Further, refiners are being constrained to use more and more refractory feeds in hydrotreatment processes. These feeds thus require catalysts which have hydrodesulphurization and hydrogenation functions which are substantially improved compared with traditional catalysts.

Finally, in contrast to other hydrotreatment processes, the hydrodesulphurization of gasolines obtained by cracking, either catalytic or non-catalytic, has to comply with two contrasting constraints: provide intense hydrodesulphurization of gasolines, while limiting hydrogenation of the unsaturated compounds (olefins) present. The hydrogenation of olefins present in ex-cracking gasolines brings about a very large drop in the octane number. Thus, catalysts have to be found which are highly selective for the hydrodesulphurization of sulphur-containing compounds and which minimize the hydrogenation of olefins.

Conventional hydrotreatment catalysts generally comprise an oxide support and an active phase based on metals from groups VIE and VIII in their oxide forms, as well as phosphorus. The preparation of such catalysts generally comprises a step for the impregnation of metals and phosphorus onto the support, followed by drying and calcining in order to obtain the active phase in its oxide form. Before using them in a hydrotreatment and/or hydrocracking reaction, such catalysts generally undergo a sulphurization step in order to form the active species which is a transition metal sulphide.

A number of avenues have been explored in order to improve the performances of such catalysts. Among them, adding an organic compound to the hydrotreatment catalysts in order to improve their activity has been recommended by the person skilled in the art, in particular for catalysts which have been prepared by impregnation followed by drying without subsequent calcining.

A number of documents describe the use of various series of organic compounds acting as additives, such as organic compounds containing nitrogen and/or organic compounds containing oxygen.

One family of compounds which is now well known in the literature concerns the chelating nitrogen compounds (EP 0 181 035, EP 1 043 069 and U.S. Pat. No. 6,540,908) with, by way of example, ethylenediaminetetraacetic acid (EDTA), ethylenediamine, diethylenetriamine or nitrilotriacetic acid (NTA).

In the oxygen-containing organic compound family, the use of mono-, di- or poly-alcohols, which are optionally etherified, has been described in the documents WO96/41848, WO01/76741, U.S. Pat. Nos. 4,012,340, 3,954,673, EP 601 722, and WO2005/035691. The prior art mentions additives comprising ester functions less frequently (EP 1 046 424, WO2006/077326).

In addition, a number of patents can be found which claim the use of carboxylic acids (EP 1 402 948, EP 0 482 817).

The document US2014/0305842 describes the use of heterocyclic compounds containing oxygen or nitrogen in the ring, such as lactams, oxacycloalkanes or lactones.

The document US2012/0205292 describes the use of compounds containing oxygen and nitrogen, such as aminocarboxylic acids or aminoalcohols.

The document WO 2014/056846 A1 describes polymerized additives. Monomers are brought into contact with the support and polymerized with the aid of an initiator and/or by increasing the temperature, before or after impregnating metals from groups VIE and VIII onto the support. Using polymers appears to improve the dispersion of the active phase.

However, to our knowledge, none of the documents pertaining to additive-containing catalysts describes the use of a catecholamine.

### SUMMARY

The aim of the invention is to provide a catalyst which has improved catalytic performances.

More particularly, the invention concerns a catalyst comprising a support based on alumina or silica or silica-alumina, at least one element selected from group VIII and/or group VIB, and at least one catecholamine.

In fact, the Applicant has established that the use of a catecholamine on a catalyst containing at least one element selected from group VIII and/or group VIB means that a hydrotreatment and/or hydrocracking catalyst can be obtained which exhibits improved catalytic performances, in particular an increase in the catalytic activity and/or an increase in the selectivity.

Typically, because of the increase in activity, the temperature necessary to obtain a desired sulphur or nitrogen content (for example 10 ppm of sulphur in the case of a diesel in the ULSD or Ultra Low Sulfur Diesel category) can be reduced.

Increasing the hydrodesulphurization selectivity (compared with the hydrogenation of olefins) is by itself particularly important in an application for the hydrodesulphurization of gasolines from catalytic cracking.

Similarly, the process for the preparation of a catalyst based on a catecholamine has the advantage of not requiring a chemical initiator for polymerization when it takes place.

In accordance with a variation, the catecholamine is selected from dopamine, noradrenaline, adrenaline and isoprenaline, alone or as a mixture. In accordance with a preferred variation, the catecholamine is dopamine.

In accordance with a variation, the content of the element from group VIB is in the range 5% to 40% by weight, expressed as the oxide of the metal from group VIB with respect to the total weight of catalyst, and the content of the element from group VIII is in the range 1% to 10% by weight, expressed as the oxide of the metal from group VIII with respect to the total weight of catalyst.

In accordance with a variation, the catalyst furthermore contains phosphorus, the quantity of phosphorus being in the range 0.01% to 20% by weight, expressed as P₂O₅ with respect to the total weight of catalyst, and the ratio of phosphorus to the element from group VIB in the catalyst being greater than or equal to 0.01.

In accordance with a variation, the quantity of catecholamine is in the range 1% to 40% by weight with respect to the weight of the support.

In accordance with a variation, the catalyst furthermore contains an organic compound other than catecholamine, containing oxygen and/or nitrogen and/or sulphur.

In accordance with this variation, the organic compound is selected from a compound comprising one or more chemical functions selected from a carboxyl, alcohol, thiol, thioether, sulphone, sulphoxide, ether, aldehyde, ketone, ester, carbonate, amine, nitrile, imide, oxime, urea and amide function.

In accordance with a variation, the catalyst is at least partially sulphurized.

The invention also concerns the process for the preparation of said catalyst, comprising the following steps:
a) bringing at least one component of an element from group VIB and/or at least one component of an element from group VIII, at least one catecholamine and optionally phosphorus, into contact with a support based on alumina or silica or silica-alumina, in a manner such as to obtain a catalyst precursor,
b) drying said catalyst precursor obtained from step a) at a temperature of less than 200° C., without subsequently calcining it.

In accordance with a preferred variation, the catecholamine is dopamine.

In accordance with a variation, step a) comprises the following steps:
a1) preparing a support comprising a catecholamine,
a2) impregnating the support obtained in step a1) with an impregnation solution comprising at least one element from group VIB and/or at least one element from group VIII and optionally phosphorus in a manner such as to obtain a catalyst precursor.

In accordance with this variation, in step a1), the support comprising a catecholamine is prepared by introducing a catecholamine at any time during the preparation of the support, and preferably during shaping of the support, or by impregnation onto a support which has already been shaped.

In accordance with another variation, step a) comprises the following steps:
a1′) bringing a solution containing at least one element from group VIB and/or at least one element from group VIII, at least one catecholamine and optionally phosphorus into contact, by co-impregnation, with a support based on alumina or silica or silica-alumina in a manner such as to obtain a catalyst precursor.

In accordance with another variation, step a) comprises the following steps:
a1″) impregnating a support based on alumina or silica or silica-alumina with at least one solution containing at least one element from group VIB and/or at least one element from group VIII and optionally phosphorus in order to obtain an impregnated support,
a2″) drying the impregnated support obtained in step a1″) at a temperature of less than 200° C. in order to obtain a dried impregnated support, and optionally calcining the dried impregnated support in order to obtain a calcined impregnated support,
a3″) impregnating the dried and optionally calcined impregnated support obtained in step a2″) with an impregnation solution comprising a catecholamine in a manner such as to obtain a catalyst precursor.

The invention also concerns the use of the catalyst in accordance with the invention or prepared in accordance with the preparation process in accordance with the invention in a process for the hydrotreatment and/or hydrocracking of hydrocarbon cuts.

Hereinbelow, the chemical element groups are given in accordance with the CAS classification (CRC Handbook of Chemistry and Physics, published by CRC press, Editor-in-chief D. R. Lide, 81^{st} edition, 2000-2001). As an example, group VIII in the CAS classification corresponds to metals from columns 8, 9 and 10 of the new IUPAC classification.

The term “hydrotreatment” means reactions which in particular encompass hydrodesulphurization (HDS), hydrodenitrogenation (HDN), hydrodeoxygenation (HDO) and the hydrogenation of aromatics (HDA).

### DETAILED DESCRIPTION OF THE INVENTION

### Catalyst

The catalyst in accordance with the invention comprises a support based on alumina or silica or silica-alumina, at least one element selected from group VIII and/or group VIB, and at least one catecholamine.

The hydrogenating, desulphurizing and denitrogenating function of said catalyst, also known as the active phase, is ensured by at least one element from group VIB and/or by at least one element from group VIII. Preferably, the catalyst in accordance with the invention comprises at least one element from group VIB and at least one element from group VIII.

The preferred elements from group VIB are molybdenum and tungsten. The preferred elements from group VIII are non-noble elements, and in particular cobalt and nickel. Advantageously, the active phase is selected from the group formed by combinations of the elements cobalt-molybdenum, nickel-molybdenum, nickel-tungsten or nickel-cobalt-molybdenum, or nickel-molybdenum-tungsten.

The total content for the elements from group VIB and group VIII is advantageously more than 6% by weight, expressed as the oxide with respect to the total weight of catalyst.

The content for the element from group VIB is in the range 5% to 40% by weight, preferably in the range 8% to 35% by weight, and more preferably in the range 10% to 30% by weight, expressed as the oxide of the metal from group VIB with respect to the total weight of catalyst.

The content of the element from group VIII is in the range 1% to 10% by weight, preferably in the range 1.5% to 9% by weight, and more preferably in the range 2% to 8% by weight, expressed as the oxide of the metal from group VIII with respect to the total weight of catalyst.

The molar ratio of the element from group VIII to the element from group VIB in the catalyst is preferably in the range 0.1 to 0.8, preferably in the range 0.15 to 0.6 and yet more preferably in the range 0.2 to 0.5.

The catalyst in accordance with the invention may also comprise phosphorus acting as a dopant. The dopant is an added element which in itself has no catalytic character, but which increases the catalytic activity of the active phase.

When phosphorus is present, the quantity of phosphorus in said catalyst is preferably in the range 0.01% to 20% by weight, expressed as P₂O₅, preferably in the range 0.01% to 15% by weight, expressed as P₂O₅, and more preferably in the range 0.02% to 10% by weight, expressed as P₂O₅.

When phosphorus is present, the molar ratio of phosphorus to the element from group VIB in the catalyst is greater than or equal to 0.01, preferably greater than or equal to 0.05, preferably in the range 0.05 and 1, and more preferably in the range 0.06 and 0.5.

The catalyst in accordance with the invention comprises a support based on alumina or silica or silica-alumina.

When the support for said catalyst is based on alumina, it contains more than 50% of alumina with respect to the weight of the support and, preferably, it contains only alumina. The alumina may be present in a crystallographic form of the gamma, delta, theta or alpha alumina type, used alone or as a mixture.

The alumina support advantageously has a total pore volume in the range 0.1 to 2 cm³.g⁻¹, preferably in the range 0.4 to 1.5 cm³.g⁻¹. The total pore volume is measured by mercury porosimetry in accordance with ASTM standard D4284 with a wetting angle of 140°, as described in the work by Rouquerol F.; Rouquerol J.; Singh K. “Adsorption by Powders & Porous Solids: Principle, methodology and applications”, Academic Press, 1999, for example using an Autopore III™ instrument from Microméritics™.

The specific surface area of the alumina is advantageously in the range 5 to 400 m².g⁻¹, preferably in the range 10 to 350 m².g⁻¹, more preferably in the range 40 to 350 m².g⁻¹. In the context of the present invention, the specific surface area is determined by the B.E.T method in accordance with ASTM standard D3663; this method is described in the work cited above.

In another preferred case, the support for said catalyst is a silica-alumina. In this case, the support based on silica-alumina contains at least 50% by weight of alumina with respect to the weight of the support. The silica content of the support is at most 50% by weight with respect to the weight of the support, usually 45% by weight or less, preferably 40% or less.

The sources of silicon are well known to the person skilled in the art. Examples which may be cited are silicic acid, silica in powder form or in the colloidal form (silica sol), and tetraethylorthosilicate Si(OEt)₄.

In another preferred case, the support is based on silica. In this case, it contains more than 50% by weight of silica with respect to the weight of the support and, in general, it contains silica alone.

In accordance with a particularly preferred variation, the support is constituted by alumina, silica or silica-alumina.

The support may also advantageously further contain 0.1% to 50% by weight of zeolite with respect to the weight of support. In this case, any source of zeolite and any of the associated preparation methods known to the person skilled in the art may be incorporated. Preferably, the zeolite is selected from the group FAU, BEA, ISV, IWR, IWW, MEI, UWY, and more preferably, the zeolite is selected from the group FAU and BEA, such as Y and/or beta zeolite.

In certain particular cases, the support may also contain at least a portion of the VIB and/or VIII metal(s), and/or at least a portion of the phosphorus when it is present, and/or at least a portion of the catecholamine which have been introduced outside of the impregnations (for example introduced during the preparation of the support, for example by co-mixing).

The support may advantageously be in the form of beads, extrudates (cylinders or multilobes, for example trilobes or quadrilobes), pellets, or irregular and non-spherical agglomerates with a specific shape which may result from a crushing step.

The catalyst in accordance with the invention also comprises at least one catecholamine.

The catecholamine is advantageously selected from dopamine, noradrenaline, adrenaline and isoprenaline, alone or as a mixture, having the following formulae:

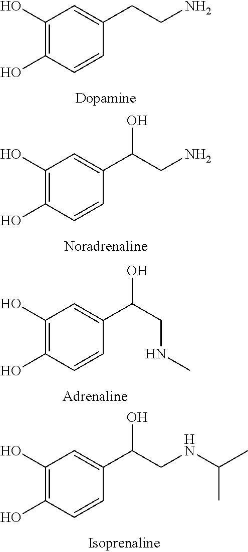

Preferably, the catalyst comprises dopamine. For solubility reasons, in particular in the impregnation solution, the dopamine is preferably used in the form of its hydrochloride, which has the following formula:

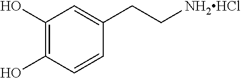

The catecholamine, and in particular the dopamine, may be present in the catalyst in the at least partially polymerized form. Without wishing to be bound by a particular theory, it would appear that the catecholamine has a tendency to cyclise into an indole type derivative (by an oxidation reaction, Acc. Chem. Res. 2010, 43, 1452) during contact with the support and then form oligomers and/or polymers, in particular during the drying step in the preparation process described below. The literature proposes two oligomerization or polymerization mechanisms: aggregation of molecules via weak bonds or true polymerization by the formation of covalent bonds between two molecules (Adv. Funct. Mater. 2012, 22, 4711., Adv. Funct. Mater. 2013, 23, 1331).

The presence of at least partially polymerized catecholamine can be detected by thermogravimetric analysis (TGA), infrared spectroscopy, UV spectroscopy, or in fact NMR spectroscopy.

The total content of the catecholamine introduced into the catalyst in accordance with the invention is in the range 1% to 40% by weight, preferably in the range 3% to 30% by weight with respect to the weight of the support. It should be noted that the quantity of catecholamine introduced is expressed with respect to the weight of the support, while the quantity of metal is expressed as the oxide, with respect to the weight of the catalyst after loss on ignition, i.e. after calcining at at least 500° C., which eliminates water and organic material. It should also be noted that this calcining is carried out in order to determine the metal content, but the catalyst in accordance with the invention is in fact a dried catalyst containing at least part of the catecholamine introduced after drying and prepared without subsequent calcining. Unless indicated otherwise, and in the case of dopamine, the content is with reference to the molecule without hydrochloride.

During the preparation of the catalyst, the drying step or steps following introduction of the catecholamine is (are) carried out at a temperature of less than 200° C. in order, preferably, to retain at least 30%, preferably at least 50%, and more preferably at least 70% of the quantity of catecholamine introduced, calculated on the basis of the carbon remaining on the catalyst.

In addition to the dopamine, the catalyst in accordance with the invention may also comprise another organic compound or a group of organic compounds known to act as additives. The function of the additives is to increase the catalytic activity compared with catalysts without additives. More particularly, the catalyst in accordance with the invention may also comprise one or more organic compounds containing oxygen and/or nitrogen and/or sulphur.

In general, the organic compound is selected from a compound comprising one or more chemical functions selected from a carboxyl, alcohol, thiol, thioether, sulphone, sulphoxide, ether, aldehyde, ketone, ester, carbonate, amine, nitrile, imide, oxime, urea and amide function.

The organic compound containing oxygen may be one or more selected from compounds comprising one or more chemical functions selected from a carboxyl, alcohol, ether, aldehyde, ketone, ester or carbonate function. By way of example, the organic compound containing oxygen may be one or more selected from the group constituted by ethylene glycol, diethylene glycol, triethylene glycol, a polyethylene glycol (with a molecular weight in the range 200 to 1500 g/mol), propylene glycol, 2-butoxyethanol, 2-(2-butoxyethoxy)ethanol, 2-(2-methoxyethoxy)ethanol, triethyleneglycol dimethylether, glycerol, acetophenone, 2,4-pentanedione, pentanone, acetic acid, maleic acid, malic acid, malonic acid, malic acid, oxalic acid, gluconic acid, tartaric acid, citric acid, gamma-ketovaleric acid, a C1-C4 dialkyl succinate, methyl acetoacetate, a lactone, dibenzofuran, a crown ether, orthophthalic acid, glucose and propylene carbonate.

The organic compound containing nitrogen may be one or more selected from compounds comprising one or more chemical functions selected from an amine or nitrile function. By way of example, the organic compound containing nitrogen may be one or more selected from the group constituted by ethylenediamine, diethylenetriamine, hexamethylenediamine, triethylenetetramine, tetraethylenepentamine, pentaethylenehexamine, acetonitrile, octylamine, guanidine or a carbazole.

The organic compound containing oxygen and nitrogen may be one or more selected from compounds comprising one or more chemical functions selected from a carboxylic acid, alcohol, ether, aldehyde, ketone, ester, carbonate, amine, nitrile, imide, amide, urea or oxime function. By way of example, the organic compound containing oxygen and nitrogen may be one or more selected from the group constituted by 1,2-cyclohexanediaminetetraacetic acid, monoethanolamine (MEA), N-methylpyrrolidone, dimethylformamide, ethylenediaminetetraacetic acid (EDTA), alanine, glycine, nitrilotriacetic acid (NTA), N-(2-hydroxyethyl)ethylenediamine-N,N′,N′-triacetic acid (HEDTA), diethylene-triaminepentaacetic acid (DTPA), tetramethylurea, glutamic acid, dimethylglyoxime, bicine or tricine, or in fact a lactam.

The organic compound containing sulphur may be one or more selected from compounds comprising one or more chemical functions selected from a thiol, thioether, sulphone or sulphoxide function. By way of example, the organic compound containing sulphur may be one or more selected from the group constituted by thioglycolic acid, 2-hydroxy-4-methylthiobutanoic acid, a sulphone derivative of a benzothiophene or a sulphoxide derivative of a benzothiophene.

When present, the quantity of organic compounds with an additive function containing oxygen and/or nitrogen and/or sulphur on the catalyst in accordance with the invention is in the range 1% to 30% by weight, preferably in the range 1.5% to 25% by weight, and more preferably in the range 2% to 20% by weight with respect to the total weight of catalyst.

### Preparation Process

The catalyst in accordance with the invention may be prepared in accordance with any mode of preparation of a supported catalyst comprising an organic compound known to the person skilled in the art.

The catalyst in accordance with the invention may be prepared in accordance with a preparation process comprising the following steps:
a) bringing at least one component of an element from group VIB and/or at least one component of an element from group VIII, at least one catecholamine and optionally phosphorus, into contact with a support based on alumina or silica or silica-alumina, in a manner such as to obtain a catalyst precursor,
b) drying said catalyst precursor obtained from step a) at a temperature of less than 200° C., without subsequently calcining it.

It is important to point out that during its preparation process, the catalyst in accordance with the invention does not undergo calcining after introducing the catecholamine or any other organic compound containing oxygen and/or nitrogen and/or sulphur when it is present, in order to preserve at least a portion of the catecholamine or any other organic compound in the catalyst. The term “calcining” as used here means a heat treatment under a gas containing air or oxygen at a temperature of 200° C. or higher.

However, the catalyst precursor may undergo a calcining step before introducing the catecholamine (or any other organic compound containing oxygen and/or nitrogen and/or sulphur), in particular after impregnation of the elements from group VIB and/or VIII (post-addition), optionally in the presence of phosphorus. Thus, the catalytic precursor may be a fresh catalyst precursor or a catalyst precursor after regeneration of a spent catalyst. In this case, the hydrogenating function comprising the elements from group VIB and/or group VIII of the catalyst in accordance with the invention, also known as the active phase, is then in an oxide form.

In accordance with another variation, the catalyst precursor does not undergo a calcining step after impregnation of the elements from group VIB and/or VIII (post-additive addition); it is merely dried. In this case, the hydrogenating function comprising the elements from group VIB and/or group VIII of the catalyst in accordance with the invention are not in an oxide form.

The elements from group VIB and/or group VIII may be introduced by any method known to the person skilled in the art. They are generally introduced by impregnation, preferably by dry impregnation or by impregnation using an excess of solution. “Dry impregnation” means that the volume of the impregnation solution corresponds exactly to the pore volume of the support; this volume is determined in advance. Preferably, the entirety of the elements from group VIB and group VIII is introduced by impregnation, preferably by dry impregnation and irrespective of the way in which it is carried out.

The elements from group VIB and/or group VIII may also be introduced at least in part during shaping of said support at the time of mixing with at least one alumina selected as the matrix; the optional remainder of the elements then being introduced subsequently by impregnation. It is also possible to introduce one of the elements from group VIB or group VIII during shaping of said support at the time of mixing, for example the element from group VIB, then to introduced the other element subsequently by impregnation, for example the element from group VIII.

The molybdenum precursors which may be used are well known to the skilled person. As an example, from among the molybdenum sources, it is possible to use oxides and hydroxides, molybdic acids and their salts, in particular ammonium salts such as ammonium molybdate, ammonium heptamolybdate, phosphomolybdic acid (H₃PMo₁₂O₄₀), and their salts, and optionally silicomolybdic acid (H₄SiMo₁₂O₄₀) and its salts. The molybdenum sources may also be any heteropolycompound of the Keggin, lacunary Keggin, substituted Keggin, Dawson, Anderson or Strandberg type, for example. Preferably, molybdenum trioxide and heteropolyanions of the Strandberg, Keggin, lacunary Keggin or substituted Keggin and phosphomolybdic type are used.

The tungsten precursors which may be used are also well known to the skilled person. As an example, from among the tungsten sources, it is possible to use oxides and hydroxides, tungstic acids and their salts, in particular ammonium salts such as ammonium tungstate, ammonium metatungstate, phosphotungstic acid and their salts, and optionally silicotungstic acid (H₄SiW₁₂O₄₀) and its salts. The sources of tungsten may also be any heteropolycompound of the Keggin, lacunary Keggin, substituted Keggin or Dawson type, for example. Preferably, oxides and ammonium salts such as ammonium metatungstate or heteropolyanions of the Keggin, lacunary Keggin or substituted Keggin or phosphotungstic acid are used.

The precursors of the elements from group VIII which may be used are advantageously selected from oxides, hydroxides, hydroxycarbonates, carbonates and nitrates of the elements from group VIII; for example, nickel hydroxycarbonate or cobalt carbonate or hydroxide are preferably used.

The phosphorus, when it is present, may advantageously be introduced in its entirety or in part, alone or as a mixture, with at least one of the elements from group VIE and group VIII, during any of the steps for impregnation of the hydrogenating function if this is introduced all at once. Said phosphorus may also be introduced in its entirety or in part during impregnation of the catecholamine if this is introduced separately from the hydrogenating function (case of post- and pre-additive addition described below) and in the presence or absence of an organic compound other than catecholamine.

It may also be introduced right from synthesis of the support, at any step in the synthesis thereof. It may also be introduced before, during or after mixing the matrix of the selected support.

The preferred phosphorus precursor is orthophosphoric acid, H₃PO₄, but its salts and esters such as ammonium phosphates are also suitable. The phosphorus may also be introduced at the same time as the element(s) from group VIB in the form of Keggin, lacunary Keggin, substituted Keggin or Strandberg type heteropolyanions.

The catecholamine is advantageously introduced by impregnation. Depending on the mode of preparation, the impregnation solution may be the same solution or a solution which differs from that containing the elements from group VIE and/or VIII.

In the case of dopamine, it is preferably introduced in the form of its hydrochloride.

In order to avoid possible polymerization of the catecholamine in the impregnation solution, the impregnation solution is advantageously acidic. Preferably, it has a pH in the range 1 to 9, preferably in the range 5 to 7.

The catecholamine may also be introduced from synthesis of the support, at any step of the synthesis thereof. It may therefore be introduced before, during or after mixing the selected matrix of the support, optionally in the presence of one or more element(s) from group VIB and/or VIII and optionally in the presence of phosphorus when it is present.

Any impregnation solution described in the present invention may comprise any polar solvent known to the person skilled in the art. Said polar solvent used is advantageously selected from polar and aprotic solvents, in particular from the group formed by methanol, ethanol and water. A list of the usual polar solvents and their dielectric constants may be found in the book “Solvents and Solvent Effects in Organic Chemistry” C. Reichardt, Wiley-VCH, 3^{rd} edition, 2003, pages 472-474.

When the catalyst additionally comprises a supplemental additive (in addition to the catecholamine) or a supplemental group of additives selected from an organic compound containing oxygen and/or nitrogen and/or sulphur, this may be introduced via an impregnation solution or in fact right from the start of synthesis of the support by mixing.

Advantageously, after each impregnation step, the impregnated support is allowed to mature. Maturation allows the impregnation solution to become homogeneously dispersed within the support.

The maturation step is advantageously carried out at atmospheric pressure, in an atmosphere saturated with water and at a temperature in the range 17° C. to 50° C., preferably at ambient temperature. In general, a maturation period in the range ten minutes to forty-eight hours and preferably in the range thirty minutes to five hours, is sufficient. Longer periods are not excluded, but do not necessarily bring about an improvement.

### Step a): A Variety of Modes of Contacting

The introduction of catecholamine in step a) may be carried out in a variety of modes which are primarily distinguished by the mode of introducing the additive which may be carried out either before impregnation of the metals (pre-additive addition), or at the same time as the metals are introduced (co-additive addition), or finally after impregnation of the metals (post-additive addition). Furthermore, the contact step a) may combine at least two modes, for example co-additive addition and post-additive addition. These various modes will be described below. Each mode, taken alone or in combination, may be implemented in one or more steps.

### Pre-Additive Addition

In accordance with a first embodiment, the contact of step a) of the process for the preparation of the catalyst in accordance with the invention comprises the following steps:
a1) preparing a support comprising a catecholamine,
a2) impregnating the support obtained in step a1) with an impregnation solution comprising at least one element from group VIB and/or at least one element from group VIII and optionally phosphorus in a manner such as to obtain a catalyst precursor.

In step a1) of the pre-impregnation embodiment, a support comprising catecholamine is prepared. The catecholamine may be introduced at any time during the preparation of the support, and preferably during shaping of the support (co-mixing) or by impregnation onto a support which has already been shaped.

If the catecholamine is to be introduced onto the already shaped support by impregnation, a solution containing catecholamine is prepared in a polar solvent, preferably water, preferably at a temperature between 15° C. and 60° C. The pH of the solution is between 1 and 9, and preferably between 5 and 7. The solution is generally stirred, advantageously for a period of 5 to 10 min. The solution is then impregnated onto the support, preferably by dry impregnation.

The impregnation step will then be followed by a step for drying at a temperature of less than 200° C., preferably in the range 70° C. to 120° C., preferably in the range 80° C. to 100° C., under drying conditions that are described below for step b). The carbon content of the dried support is in the range 2% to 12% by weight.

If introduction is to be carried out during shaping of the support, an alumina or silica-alumina or silica powder is mixed with a solution, preferably aqueous, containing the catecholamine and optionally with a binder and a peptising agent (for example nitric acid). The mixture is homogenized in a mixer. After having obtained an intimate and homogeneous mixture, shaping is carried out by extrusion, by pelletization, by the oil-drop method, by rotary plate granulation or by any method which is well known to the person skilled in the art. Highly preferably, said shaping is carried out by extrusion.

The shaping step will then be followed by drying at a temperature of less than 200° C., preferably in the range 70° C. to 120° C., preferably in the range 80° C. to 100° C., under drying conditions such as those which are described below in step b). The carbon content of the dried support is in the range 2% to 12% by weight.

In step a2) of the pre-impregnation embodiment, the elements from group VIB and/or group VIII and optional phosphorus may advantageously be introduced via one or more impregnations of an excess of solution onto the support, or, as is preferable, via one or more dry impregnations, and preferably by a single dry impregnation of said support, using solution(s), preferably aqueous solution(s), containing the precursor or precursors of the metals and optionally the phosphorus precursor.

When phosphorus or a supplemental additive (in addition to the catecholamine) or a group of supplemental additives selected from an organic compound containing oxygen and/or nitrogen and/or sulphur are to be introduced, introduction may be carried out onto the support of step a1) during shaping or by impregnation, and/or into the impregnation solution of step a2), or in fact via a supplemental impregnation at any point in the preparation process before final drying in step b).

### Co-Additive Addition

In this embodiment, the catecholamine and the components of the elements from group VIB and/or group VIII are introduced simultaneously onto said support.

In accordance with this embodiment, step a) is the following step:
a1′) bringing a solution containing at least one element from group VIB and/or at least one element from group VIII, at least one catecholamine and optionally phosphorus into contact, by co-impregnation, with a support based on alumina or silica or silica-alumina in a manner such as to obtain a catalyst precursor.

The co-impregnation step or steps is(are) preferably carried out by dry impregnation or by impregnation with an excess of solution. When this embodiment comprises using several co-impregnation steps, each co-impregnation step is preferably followed by an intermediate drying step at a temperature of less than 200° C., advantageously in the range 50° C. to 180° C., preferably in the range 70° C. to 150° C., highly preferably in the range 75° C. to 130° C. and optionally with a maturation period between the impregnation step and the drying step.

In accordance with a variation of the co-additive addition, initially, a compound is formed from an element from group VIB and a catecholamine by bringing a solution containing an element from group VIB, preferably molybdenum, and a solution containing the catecholamine into contact under conditions in which a precipitate is formed which contains the element from group VIB and the catecholamine. The compound is mixed with the alumina or silica-alumina or silica powder, with water and optionally with a binder and a peptising agent (for example nitric acid). The mixture is homogenized in a mixer. After having obtained an intimate and homogeneous mixture, shaping is carried out by extrusion, by pelletization, by the oil-drop method, by rotating plate granulation or by any method which is well known to the person skilled in the art. Highly preferably, said shaping is carried out by extrusion.

The shaping step will then be followed by drying at a temperature of less than 200° C., preferably in the range 70° C. to 120° C., preferably in the range 80° C. to 100° C., under drying conditions such as those which are described below in step b). The carbon content of the dried support is in the range 2% to 12% by weight.

The element from group VIII may be added during the co-mixing step or subsequently by impregnation or by any other method known to the person skilled in the art.

### Post-Additive Addition

In accordance with a third embodiment of step a) of the process for the preparation of the catalyst in accordance with the invention (post-additive addition), at least the catecholamine is brought into contact with a dried and optionally calcined impregnated support comprising at least one component of an element from group VIB and/or at least one component of an element from group VIII, and optionally phosphorus, said support being based on alumina or silica or silica-alumina, in a manner such as to obtain a catalyst precursor.

In accordance with this third embodiment, the contact of step a) comprises the following successive steps which will be detailed as follows:
a1″) impregnating a support based on alumina or silica or silica-alumina with at least one solution containing at least one element from group VIB and/or at least one element from group VIII and optionally phosphorus in order to obtain an impregnated support,
a2″) drying the impregnated support obtained in step a1″) at a temperature of less than 200° C. in order to obtain a dried impregnated support, and optionally calcining the dried impregnated support in order to obtain a calcined impregnated support,
a3″) impregnating the dried and optionally calcined impregnated support obtained in step a2″) with an impregnation solution comprising a catecholamine in a manner such as to obtain a catalyst precursor.

In step a1″) of the post-impregnation embodiment, the elements from group VIB and/or group VIII and optionally phosphorus may advantageously be introduced onto the support by means of one or more impregnations of an excess of solution onto the support or, as is preferable, via one or more dry impregnations, and, preferably, by a single dry impregnation onto said support, with the aid of solution(s), preferably aqueous, containing the metal precursor or precursors and preferably the phosphorus precursor.

In accordance with another variation, the elements from group VIB and/or group VIII and optionally phosphorus, and an optional organic compound other than catecholamine, may be introduced in step a1″) in a successive manner using several impregnation solutions containing one or more of the components.

When several impregnation steps are carried out, each impregnation step is preferably followed by an intermediate drying step at a temperature of less than 200° C., advantageously in the range 50° C. to 180° C., preferably in the range 70° C. to 150° C., highly preferably in the range 75° C. to 130° C., and optionally a maturation period is carried out between impregnation and drying.

In accordance with step a2″), the impregnated support obtained in step a1″) is dried at a temperature of less than 200° C. in order to obtain a dried impregnated support under the conditions described for drying in step b) below.

Optionally, the dried impregnated support may then undergo calcining. Calcining is generally carried out at a temperature in the range 200° C. to 900° C., preferably in the range 250° C. to 750° C. The calcining period is generally in the range 0.5 hours to 16 hours, preferably in the range 1 hour to 5 hours. It is generally carried out in air. Calcining can be used to transform the precursors of the metals from groups VIB and VIII into oxides.

In accordance with step a3″), the dried impregnated support obtained in step a2″) is impregnated with an impregnation solution comprising the catecholamine in a manner such as to obtain a catalyst precursor.

The catecholamine may advantageously be deposited in one or more steps, either by excess impregnation, or by dry impregnation, or using any other means known to the person skilled in the art. Preferably, the catecholamine is introduced by dry impregnation, in the presence of a solvent as described above. Preferably, the solvent in the impregnation solution employed in step a3″) is water, which facilitates its use on an industrial scale.

### Step b): Drying Without Subsequent Calcining

The catalyst precursor obtained by pre-, co-, post-additive addition, or in fact a mixture of these variations, then undergoes the drying step b).

In accordance with step b) of the preparation process in accordance with the invention, the catalyst precursor obtained in step a), optionally matured, undergoes a step for drying at a temperature of less than 200° C. without a subsequent calcining step.

Any step for drying after introducing the catecholamine or any other additives is carried out at a temperature of less than 200° C., preferably in the range 50° C. to 180° C., more preferably in the range 70° C. to 150° C. and highly preferably in the range 75° C. to 130° C.

The drying step is advantageously carried out using any technique which is known to the person skilled in the art. It is advantageously carried out at atmospheric pressure or under reduced pressure. Preferably, this step is carried out at atmospheric pressure. It is advantageously carried out in a flushed bed using air or any other hot gas. Preferably, when drying is carried out in a fixed bed, the gas used is either air or an inert gas such as argon or nitrogen. Highly preferably, drying is carried out in a flushed bed in the presence of nitrogen and/or air. Preferably, the drying step is of short duration lasting in the range 5 minutes to 12 hours, preferably in the range 30 minutes to 6 hours and highly preferably in the range 1 hour to 3 hours.

At the end of the drying step b), a dried catalyst is obtained which does not undergo any subsequent calcining step.

### Sulphurization

Before using it for the hydrotreatment and/or hydrocracking reaction, it is advantageous to transform the dried catalyst obtained in accordance with any one of the catalyst preparation embodiments described in the present invention into a sulphurized catalyst in order to form its active species. This step for activation or sulphurization is carried out using methods which are well known to the person skilled in the art, and advantageously under a sulpho-reducing atmosphere in the presence of hydrogen and hydrogen sulphide.

At the end of step b) in the various embodiments for the preparation in accordance with the invention, said catalyst obtained thus advantageously undergoes a sulphurization step without an intermediate calcining step.

Said dried catalyst is advantageously sulphurized in an ex situ or in situ manner. The sulphurizing agents are H₂S gas or any other compound containing sulphur used for the activation of hydrocarbon feeds with a view to sulphurizing the catalyst. Said compounds containing sulphur are advantageously selected from alkyl disulphides such as, for example, dimethyldisulphide (DMDS), alkyl sulphides such as dimethyl sulphide, for example, thiols such as n-butylmercaptan (or 1-butanethiol), for example, polysulphide compounds of the tertiononoylpolysulphide type, or any other compound which is known to the person skilled in the art and can be used to obtain good sulphurization of the catalyst. Preferably, the catalyst is sulphurized in situ in the presence of a sulphurizing agent and a hydrocarbon feed. Highly preferably, the catalyst is sulphurized in situ in the presence of a hydrocarbon feed supplemented with dimethyldisulphide.

Hydrotreatment and/or Hydrocracking Process

Finally, in another aspect, the invention concerns the use of the catalyst in accordance with the invention, or prepared in accordance with the preparation process in accordance with the invention, in processes for the hydrotreatment and/or hydrocracking of hydrocarbon cuts.

The catalyst in accordance with the invention which has preferably undergone a prior sulphurization step is advantageously used for the reactions for the hydrotreatment and/or hydrocracking of hydrocarbon feeds such as oil cuts, cuts obtained from coal or hydrocarbons produced from natural gas, optionally as a mixture or in fact starting from a hydrocarbon cut obtained from biomass, and more particularly for the reactions of hydrogenation, hydrodenitrogenation, hydrodearomatization, hydrodesulphurization, hydrodeoxygenation, hydrodemetallization or hydroconversion of hydrocarbon feeds.

Examples of the feeds employed in the hydrotreatment process are gasolines, gas oils, vacuum gas oils, atmospheric residues, vacuum residues, atmospheric distillates, vacuum distillates, heavy fuels, oils, waxes and paraffins, spent oils, residues or deasphalted crudes, feeds from thermal or catalytic conversion processes, lignocellulosic feeds or, more generally, feeds obtained from biomass, used alone or as a mixture. The feeds which are treated, and in particular those cited above, generally contain heteroatoms such as sulphur, oxygen and nitrogen and, for the heavy feeds, they also often contain metals.

The operating conditions used in the processes carrying out the hydrotreatment reactions of hydrocarbon feeds as described above are generally as follows: the temperature is advantageously in the range 180° C. to 450° C., and preferably in the range 250° C. to 440° C., the pressure is advantageously in the range 0.5 to 30 MPa, and preferably in the range 1 to 18 MPa, the hourly space velocity is advantageously in the range 0.1 to 20 h⁻¹ and preferably in the range 0.2 to 5 h⁻¹, and the hydrogen/feed ratio, expressed as the volume of hydrogen measured under normal temperature and pressure conditions per volume of liquid feed is advantageously in the range 50 L/L to 5000 L/L and preferably 80 to 2000 L/L.

In accordance with a first mode of use, said hydrotreatment process in accordance with the invention is a hydrotreatment process, and in particular hydrodesulphurization (HDS) of a gas oil cut carried out in the presence of at least one catalyst in accordance with the invention. Said hydrotreatment process in accordance with the invention is intended to eliminate the sulphur-containing compounds present in said gas oil cut in a manner such as to comply with the environmental specifications in force, namely an allowed sulphur content of up to 10 ppm. It can also be used to reduce the aromatics and nitrogen contents of the gas oil cut to be hydrotreated.

Said gas oil cut to be hydrotreated in accordance with the process of the invention generally contains 0.02% to 5.0% by weight of sulphur. It may be obtained from straight run distillation of crude oil, from a coking unit, from a visbreaking unit, from a steam cracking unit, from a unit for the hydrotreatment and/or hydrocracking of heavier feeds and/or from a fluid catalytic cracking unit. Said gas oil cut preferably has at least 90% by weight of compounds with a boiling temperature in the range 250° C. to 400° C. at atmospheric pressure.

The process for the hydrotreatment of said gas oil cut in accordance with the invention is carried out under the following operating conditions: a temperature in the range 200° C. to 400° C., preferably in the range 300° C. to 380° C., a total pressure in the range 2 MPa to 10 MPa and more preferably in the range 3 MPa to 8 MPa, with a ratio of the volume of hydrogen per volume of hydrocarbon feed, expressed as the volume of hydrogen, measured under normal temperature and pressure conditions, per volume of liquid feed in the range 100 to 600 litres per litre and more preferably in the range 200 to 400 litres per litre and an hourly space velocity in the range 1 to 10 h⁻¹, preferably in the range 2 to 8 h⁻¹. The HSV corresponds to the inverse of the contact time expressed in hours and is defined by the ratio of the volume flow rate of liquid hydrocarbon feed over the volume of catalyst charged into the reaction unit carrying out the hydrotreatment process in accordance with the invention. The reaction unit carrying out the process for the hydrotreatment of said gas oil cut in accordance with the invention is preferably operated in fixed bed mode, in moving bed mode or in ebullated bed mode, preferably in fixed bed mode.

In accordance with a second mode of use, said hydrotreatment and/or hydrocracking process in accordance with the invention is a process for hydrotreatment (in particular hydrodesulphurization, hydrodenitrogenation, hydrogenation of aromatics) and/or for hydrocracking a vacuum distillate cut carried out in the presence of at least one catalyst in accordance with the invention. Said hydrotreatment and/or hydrocracking process, also known as the hydrotreatment or hydrocracking pre-treatment process in accordance with the invention, depending on the case, is aimed at eliminating the sulphur-containing, nitrogen-containing or aromatic compounds present in said distillate cut in a manner such as to carry out a pre-treatment before conversion in catalytic cracking or hydroconversion processes, or to hydrocrack the distillate cut which could possibly have been pre-treated beforehand, if necessary.

Extremely varied feeds may be treated by the processes for hydrotreatment and/or hydrocracking of vacuum distillates described above. The feed may, for example, be vacuum distillates as well as feeds obtained from units for the extraction of aromatics from lubricating base oils or obtained from solvent dewaxing of lubricating base oils and/or of deasphalted oils, or in fact the feed may be a deasphalted oil or paraffins obtained from the Fischer-Tropsch process, or in fact any mixture of the feeds cited above. In general, the feeds have a T5 boiling point of more than 340° C. at atmospheric pressure, and preferably more than 370° C. at atmospheric pressure, i.e., 95% by weight of the compounds present in the feed have a boiling point of more than 340° C., preferably more than 370° C. The nitrogen content in the feeds treated in the processes in accordance with the invention is usually more than 200 ppm by weight, preferably in the range 500 to 10000 ppm by weight. The sulphur content in the feeds treated in the processes in accordance with the invention is usually in the range 0.01% to 5.0% by weight. The feed may optionally contain metals (for example nickel and vanadium). The asphaltenes content is generally below 3000 ppm by weight.

The hydrotreatment and/or hydrocracking catalyst is generally brought into contact with the feeds described above in the presence of hydrogen, at a temperature of more than 200° C., usually in the range 250° C. to 480° C., advantageously in the range 320° C. to 450° C., preferably in the range 330° C. to 435° C., under a pressure of more than 1 MPa, usually in the range 2 to 25 MPa, preferably in the range 3 to 20 MPa, the hourly space velocity being in the range 0.1 to 20.0 h⁻¹ and preferably 0.1-6.0 h⁻¹, preferably 0.2-3.0 h⁻¹, and the quantity of hydrogen introduced is such that the volume ratio in litres of hydrogen/litres of hydrocarbon, expressed as the volume of hydrogen, measured under normal temperature and pressure conditions, per volume of liquid feed, is in the range 80 to 5000 L/L and usually in the range 100 to 2000 L/L. These operating conditions used in the processes in accordance with the invention can generally be used to obtain conversions per pass into products with boiling points of less than 340° C. at atmospheric pressure, and preferably less than 370° C. at atmospheric pressure, of more than 15% and more preferably in the range 20% to 95%.

The processes for the hydrotreatment and/or hydrocracking of vacuum distillates using the catalysts in accordance with the invention cover fields of pressure and conversion ranging from mild hydrocracking to high pressure hydrocracking. The term “mild hydrocracking” means hydrocracking resulting in moderate conversions, generally below 40%, and functioning at low pressure, generally between 2 MPa and 6 MPa.

The catalyst in accordance with the invention may be used alone, in a single or in a plurality of fixed catalytic beds, in one or more reactors, in a hydrocracking process flow diagram termed a once-through process flow diagram, with or without a liquid recycle of the unconverted fraction, or in fact in a hydrocracking process flow diagram termed a two-step process flow diagram, optionally in association with a hydrorefining catalyst located upstream of the catalyst of the present invention.

In accordance with a third mode of use, said hydrotreatment and/or hydrocracking process in accordance with the invention is advantageously carried out as a pre-treatment in a fluid catalytic cracking process (FCC). The operating conditions for the pre-treatment in terms of the temperature, pressure, hydrogen recycle and hourly space velocity ranges are generally identical to those described above for the processes for the hydrotreatment and/or hydrocracking of vacuum distillates. The FCC process may be carried out in a conventional manner known to the person skilled in the art under suitable cracking conditions with a view to producing hydrocarbon products with a low molecular weight. A brief description of catalytic cracking is to be found, for example, in ULLMANS ENCYCLOPEDIA OF INDUSTRIAL CHEMISTRY VOLUME A 18, 1991, pages 61 to 64.

In accordance with a fourth mode of use, said hydrotreatment and/or hydrocracking process in accordance with the invention is a process for the hydrotreatment (in particular hydrodesulphurization) of a gasoline cut containing olefins in the presence of at least one catalyst in accordance with the invention.

In contrast to other hydrotreatment processes, the hydrotreatment (in particular hydrodesulphurization) of gasolines must be able to deal with two contrasting constraints: providing intense hydrodesulphurization of the gasolines and limiting hydrogenation of the unsaturated compounds (olefins) present in order to limit the fall in octane number.

The feed is generally a hydrocarbon cut with a distillation range in the range 30° C. to 260° C. Preferably, this hydrocarbon cut is a gasoline type cut. Highly preferably, the gasoline cut is an olefinic gasoline cut obtained, for example, from a fluid catalytic cracking unit.

The hydrotreatment process consists of bringing the hydrocarbon cut into contact with the catalyst in accordance with the invention and hydrogen under the following conditions: at a temperature in the range 200° C. to 400° C., preferably in the range 230° C. to 330° C., at a total pressure in the range 1 to 3 MPa, preferably in the range 1.5 to 2.5 MPa, at an hourly space velocity (HSV), defined as the volume flow rate of feed with respect to the volume of catalyst, in the range 1 to 10 h⁻¹, preferably in the range 2 to 6 h⁻¹, and with a hydrogen/gasoline feed volume ratio in the range 100 to 600 NL/L, preferably in the range 200 to 400 NL/L.

The process for the hydrotreatment of gasolines may be carried out in one or more reactors in series of the fixed bed or ebullated bed type. If the process is carried out using at least two reactors in series, it is possible to provide equipment for eliminating H₂S from the effluent obtained from the first hydrodesulphurization reactor before treating said effluent in the second hydrodesulphurization reactor.

The following examples demonstrate the large gain in activity or selectivity for the catalysts in accordance with the invention compared with catalysts which do not include any catecholamine.

### EXAMPLES

### Example 1A: Preparation of Catalyst Cdop1 (CoMoP/Pdop@Al₂O₃) by Pre-Additive Addition of an Al₂O₃-1 Support

A catalyst was prepared by pre-additive addition of dopamine onto an Al₂O₃-1 support followed by impregnation with CoMoP, envisaging a quantity of Mo of 20% by weight, expressed as MoO₃.
a) 1.37 grams of dopamine hydrochloride was dissolved in water in order to obtain a solution of 20 mL.
b) 20 g of an Al₂O₃-1 support (BET surface area 137 m²/g, pore volume 1 mL/g, in the form of beads from 1.4 to 2 mm) were placed in a beaker. The solution prepared in the preceding step was slowly impregnated into the support. The impregnated support was then matured in a water-saturated atmosphere for 12 h.
c) The support was then oven dried at 90° C. for 20 h to result in a support (Pdop@Al₂O₃-1). The support supplemented with additive contained 5.2% by weight of dopamine (or 6.4% by weight of dopamine hydrochloride).
d) 1.38 g of phosphomolybdic acid (H₃PMo₁₂O₄₀) and 0.873 g of Co(NO₃)₂ were dissolved in ethanol in order to obtain 3.8 mL of solution.
e) This solution was impregnated drop by drop onto 4 g of the support Pdop@Al₂O₃-1. The impregnated support was matured in an atmosphere saturated with ethanol for 12 h. It was then dried at 40° C. under vacuum for 2 h.
f) The catalyst obtained in this manner, CoMoP/Pdop@Al₂O₃-1 (Cdop1), contained 20% by weight of MoO₃, 4.4% by weight of CoO, and 0.8% by weight of P₂O₅ (expressed as the oxide). The molar ratio Co/(Co+Mo) was 0.3. The quantity of dopamine with respect to the support was 5.2% by weight (or 6.4% by weight of dopamine hydrochloride).

### Example 1B (Comparative for Example 1A): Preparation of Catalyst C1 (CoMoP/Al₂O₃)

A catalyst was prepared by impregnation with CoMoP, envisaging a content of 20% by weight of Mo, expressed as MoO₃, onto the Al₂O₃-1 support, which had no pre-addition of additive using dopamine:
a) 2.94 g of phosphomolybdic acid and 1.86 g of Co(NO₃)₂ were dissolved in ethanol in order to obtain 8.6 mL of solution.
b) This solution was impregnated drop by drop onto 8 g of the support Al₂O₃-1. The impregnated support was matured in an atmosphere saturated with ethanol for 12 h. It was then dried at 40° C. under vacuum for 2 h.
c) The catalyst obtained in this manner, CoMoP/Al₂O₃-1 (C1), contained 20% by weight of MoO₃, 4.4% by weight of CoO and 0.8% by weight of P₂O₅ (expressed as the oxide). The molar ratio Co/(Co+Mo) was 0.3.

### Example 2A: Preparation of Catalyst Cdop2 (CoMoP/Pdop@Al₂O₃) by Pre-Additive Addition of an Al₂O₃-1 Support

A catalyst was prepared by pre-additive addition of dopamine onto an Al₂O₃-1 support followed by impregnation with CoMoP, envisaging a quantity of Mo of 10% by weight, expressed as MoO₃.

The Al₂O₃-1 support was prepared by pre-additive addition of dopamine following steps a) to c) of Example 1A. Next, the following steps were carried out:
d) 0.628 g of phosphomolybdic acid (H₃PMo₁₂O₄₀) and 0.397 g of Co(NO₃)₂ were dissolved in ethanol in order to obtain 3.8 mL of solution.
e) This solution was impregnated drop by drop onto 4 g of the support Pdop@Al₂O₃-1. The impregnated support was matured in an atmosphere saturated with ethanol for 12 h. It was then dried at 40° C. under vacuum for 2 h.
f) The catalyst obtained in this manner, CoMoP/Pdop@Al₂O₃-1 (Cdop2), contained 10% by weight of MoO₃, 2.3% by weight of CoO and 0.4% by weight of P₂O₅ (with respect to the weight of oxide, i.e. after loss on ignition). The molar ratio Co/(Co+Mo) was 0.3. The quantity of dopamine with respect to the support was 5.2% by weight (or 6.4% by weight of dopamine hydrochloride).

### Example 2B (Comparative for Example 2A): Preparation of Catalyst C2 CoMoP/Al₂O₃

A catalyst was prepared by impregnation with CoMoP, envisaging a content of 10% by weight of Mo, expressed as MoO₃ onto the Al₂O₃-1 support which had no pre-addition of additive using dopamine:
a) 1.57 g of phosphomolybdic acid and 0.993 g of Co(NO₃)₂ were dissolved in ethanol in order to obtain a solution of 8.6 mL.
b) This solution was impregnated drop by drop onto 10 g of the Al₂O₃-1 support. The impregnated support was matured in an atmosphere saturated with ethanol for 12 h. It was then dried at 40° C. under vacuum for 2 h.
c) The catalyst obtained in this manner, CoMoP/Al₂O₃-1 (C2), contained 10% by weight of MoO₃, 2.2% by weight of CoO and 0.4% by weight of P₂O₅ (expressed as the oxide). The molar ratio Co/(Co+Mo) was 0.3.

### Example 3A: Preparation of Catalyst Cdop3 (CoMoP/Pdop@Al₂O₃) by Pre-Additive Addition of an Al₂O₃-2 Support

A catalyst was prepared by pre-additive addition of dopamine onto an Al₂O₃-2 support followed by impregnation with CoMoP, envisaging a quantity of Mo of 20% by weight, expressed as MoO₃, and a high dopamine content.
a) 5.52 grams of dopamine hydrochloride were dissolved in water in order to obtain 26 mL of solution.
b) 35 g of an Al₂O₃-2 support (BET surface area 265 m²/g, pore volume 0.73 mL/g, in the form of trilobal extrudates 1.6 mm in diameter) was placed in a beaker. The solution prepared in the preceding step was slowly impregnated into the support. The impregnated support was then matured in an atmosphere saturated with water for 12 h.
c) The support was then oven dried at 90° C. for 20 h to result in a support covered with partially polymerized dopamine (Pdop@Al₂O₃-2). The support contained 11.2% by weight of dopamine (or 13.8% by weight of dopamine hydrochloride).
d) 1.90 g of phosphomolybdic acid (H₃PMo₁₂O₄₀) and 1.20 g of Co(NO₃)₂ were dissolved in ethanol in order to obtain a solution of 3.8 mL.
e) This solution was impregnated drop by drop onto 6 g of the support Pdop@Al₂O₃-2. The impregnated support was matured in an atmosphere saturated with ethanol for 12 h. It was then dried at 40° C. under vacuum for 2 h.
f) The catalyst obtained in this manner, CoMoP/Pdop@Al₂O₃-2 (Cdop3), contained 20% by weight of MoO₃, 4.5% by weight of CoO and 0.8% by weight of P₂O₅ (expressed as the oxide). The molar ratio Co/(Co+Mo) was 0.3. The quantity of dopamine with respect to the support was 11.2% by weight (or 13.8% by weight of dopamine hydrochloride).

### Example 3B (Comparative for Example 3A): Preparation of Catalyst C3 (CoMoP/Al₂O₃)

A catalyst was prepared by impregnation with CoMoP, envisaging a content of 20% by weight of Mo, expressed as MoO₃, onto the Al₂O₃-2 support which had no pre-addition of additive using dopamine:
a) 2.2 g of phosphomolybdic acid and 1.86 g of Co(NO₃)₂ were dissolved in ethanol in order to obtain 4.8 mL of solution.
b) This solution was impregnated drop by drop onto 6 g of the Al₂O₃-2 support. The impregnated support was matured in an atmosphere saturated with ethanol for 12 h. It was then dried at 40° C. under vacuum for 2 h.
c) The catalyst obtained in this manner, CoMoP/Al₂O₃-2 (C3), contained 20% by weight of MoO₃, 4.5% by weight of CoO and 0.8% by weight of P₂O₅ (expressed as the oxide). The molar ratio Co/(Co+Mo) was 0.3.

### Example 4A: Preparation of Catalyst Cdop4 (CoMoP/Pdop@SiO₂)

A catalyst was prepared by pre-additive addition of dopamine onto a SiO₂ support followed by impregnation with CoMoP, envisaging a quantity of Mo, expressed as MoO₃, of 20% by weight.
a) 4.14 g of dopamine hydrochloride was dissolved in water in order to obtain 37 mL of solution.
b) 26 g of a SiO₂ support (BET surface area 233 m²/g, pore volume 1.10 mL/g, in the form of cylindrical extrudates 1.6 mm in diameter) was placed in a beaker. The solution prepared in the preceding step was slowly impregnated into the support. The impregnated support was then matured in a water-saturated atmosphere for 12 h.
c) The support was then oven dried at 90° C. for 20 h. The support contained 11% by weight of dopamine (or 13.6% by weight of dopamine hydrochloride).
d) 1.90 g of phosphomolybdic acid (H₃PMo₁₂O₄₀) and 1.20 g of Co(NO₃)₂ were dissolved in ethanol in order to obtain a solution of 6 mL.
e) This solution was impregnated drop by drop onto 6 g of the support Pdop@SiO₂. The impregnated support was matured in an atmosphere saturated with ethanol for 12 h. It was then dried at 40° C. under vacuum for 2 h.
f) The catalyst obtained in this manner, CoMoP/Pdop@SiO₂ (Cdop4), contained 20% by weight of MoO₃, 4.5% by weight of CoO and 0.8% by weight of P₂O₅ (expressed as the oxide). The molar ratio Co/(Co+Mo) was 0.3. The quantity of dopamine with respect to the support was 11% by weight (or 13.6% by weight of dopamine hydrochloride).

### Example 4B (Comparative for Example 4A): Preparation of Catalyst C4 CoMoP/SiO₂

A catalyst was prepared by impregnation with CoMoP, envisaging a content of 20% by weight of Mo, expressed as MoO₃, onto the SiO₂ support which had no pre-addition of additive using dopamine:
a) 3.67 g of phosphomolybdic acid and 2.32 g of Co(NO₃)₂ were dissolved in ethanol in order to obtain a solution of 13 mL.
b) This solution was impregnated drop by drop onto 10 g of the SiO₂ support. The impregnated support was matured in an atmosphere saturated with ethanol for 12 h. It was then dried at 40° C. under vacuum for 2 h.
c) The catalyst obtained in this manner, CoMoP/SiO₂ (C4), contained 20% by weight of MoO₃, 4.5% by weight of CoO and 0.8% by weight of P₂O₅ (expressed as the oxide). The molar ratio Co/(Co+Mo) was 0.3.

### Example 5: Preparation of Catalyst Cdop5 (CoMoP/Pdop@Al₂O₃) by Pre-Additive Addition of an Al₂O₃-3 Support by Co-Mixing

A catalyst was prepared by pre-additive addition of dopamine onto an Al₂O₃-3 support by co-mixing, followed by impregnation with CoMoP, envisaging a quantity of Mo of 20% by weight, expressed as MoO₃.

28.12 g of alumina-3 (in the form of a powder, BET surface area 279 m²/g, pore volume 1.28 mL/g), 1.13 g of methyl cellulose and 3.16 g of dopamine hydrochloride were introduced into a mixer (Brabender®). The mixture was homogenized by mixing. 0.234 g of nitric acid as a peptising agent dissolved in 29 mL of water was slowly added to the mixture, and mixing was continued until a paste with a good consistency for extrusion was obtained. The paste was then introduced into an extruder in order to produce trilobal extrudates. The support was then oven dried at 90° C. for 20 h. The support contained 8.1% by weight of dopamine (or 10% by weight of dopamine hydrochloride).

The catalyst Cdop5 was prepared by impregnation onto this support Pdop@Al₂O₃-3, in accordance with the following steps:
a) 1.98 g of phosphomolybdic acid and 1.25 g of Co(NO₃)₂ were dissolved in ethanol in order to obtain a solution of 5.28 mL.
b) This solution was impregnated drop by drop onto 6 g of the support Pdop@Al₂O₃-3. The impregnated support was matured in an atmosphere saturated with ethanol for 12 h. It was then dried at 40° C. under vacuum for 2 h.
c) The catalyst obtained in this manner, CoMoP/Al₂O₃-3 (Cdop5), contained 20% by weight of MoO₃, 4.6% by weight of CoO and 0.8% by weight of P₂O₅ (expressed as the oxide). The molar ratio Co/(Co+Mo) was 0.3. The quantity of dopamine with respect to the support was 8.1% by weight (or 10% by weight of dopamine hydrochloride).

### Example 6: Preparation of a Catalyst Cdop6 (CoMoP/Pdop@Al₂O₃) by Co-Impregnation

A catalyst was prepared by co-additive addition of dopamine, cobalt, molybdenum and phosphorus onto an Al₂O₃-1 support, envisaging a quantity of Mo of 10% by weight, expressed as MoO₃.
a) 1.57 g of phoshomolybdic acid, 0.993 g of Co(NO₃)₂ and 0.686 g of dopamine hydrochloride were dissolved in ethanol in order to obtain a solution with a volume of 10.8 mL.
b) This solution was slowly impregnated onto 10 g of Al₂O₃-1. The impregnated support was matured in an atmosphere saturated with ethanol for 12 h. It was then dried at 40° C. under vacuum for 2 h.
c) The catalyst obtained in this manner, CoMoP@Pdop@Al₂O₃-1 (Cdop6), contained 10% by weight of MoO₃, 2.2% by weight of CoO and 0.4% by weight of P₂O₅ (expressed as the oxide). The molar ratio Co/(Co+Mo) was 0.3. The quantity of dopamine with respect to the support was 5.2% by weight (or 6.4% by weight of dopamine hydrochloride).

### Example 7: Preparation of a Catalyst Cdop7 CoMo/Pdop@Al₂O₃ by Co-Additive Addition via Co-Mixing

A catalyst was prepared by precipitation of a Mo-dopamine compound, which had been co-mixed with alumina-3 and a precursor of cobalt, then dried. This catalyst in accordance with the invention did not contain phosphorus.
a) 4.82 g of Na₂MoO₄ was added to a solution of dopamine (8.87 g) dissolved in 70 mL of water. The solution was stirred overnight. Next, it was filtered under vacuum and washed with water (50 mL) and ethanol (60 mL) in order to precipitate the Mo:dopamine(1:2) complex. The precipitate obtained was oven dried under vacuum at 120° C. for 20 h. The quantity of dopamine in the complex was 70% by weight.
b) 15 g of Al₂O₃-3 (in the form of a powder), 0.71 g of methyl cellulose and 13.15 g of the Mo:dopamine (1:2) precipitate obtained in step a) were introduced into a mixer (Brabender®). The mixture was homogenized by mixing. 0.159 g of nitric acid as a peptising agent and 3.68 g of Co(NO₃)₂ dissolved in 26.6 mL of water were slowly added to the mixture and mixing was continued until a paste with a good consistency for extrusion was obtained.
c) The paste was then introduced into an extruder in order to produce trilobal extrudates (1.6 mm in diameter).
d) The extrudates were dried at 120° C. for 20 h.
e) The catalyst obtained in this manner, CoMoP@Pdop@Al₂O₃-3 (Cdop7), contained 20% by weight of MoO₃ and 4.4% by weight of CoO (expressed as the oxide). The molar ratio Co/(Co+Mo) was 0.3. The quantity of dopamine with respect to the support Al₂O₃ was 38%.

### Example 8: Preparation of a Catalyst Cdop8 (Pdop/CoMoP/Al₂O₃) by Post-Additive Addition

A catalyst was prepared by post-additive addition of dopamine onto a catalyst precursor of alumina Al₂O₃-1 containing cobalt, molybdenum and phosphorus, envisaging a quantity of Mo of 20% by weight, expressed as MoO₃.
a) 0.224 g of dopamine hydrochloride was dissolved in water in order to obtain a solution of 3.4 mL.
b) This solution was impregnated onto 5 g of a precursor of catalyst C1, followed by maturation in an atmosphere saturated with water for 12 h.
c) The extrudates were dried at 90° C. for 20 h.
d) The catalyst obtained in this manner, CoMoP/Pdop/Al₂O₃-1 (Cdop8), contained 20% by weight of MoO₃, 4.4% by weight of CoO and 0.8% by weight of P₂O₅ (expressed as the oxide). The molar ratio Co/(Co+Mo) was 0.3. The quantity of dopamine was 5.2% by weight (or 6.4% by weight of dopamine hydrochloride).

### Example 9: Catalytic Test: Hydrogenation of Toluene

The toluene hydrogenation test is intended to evaluate the hydrogenating activity of catalysts in the presence of H₂S and under hydrogen pressure.

The mass of catalysts corresponding to a bed volume of 0.45 cm³ was charged into a fixed bed flushed reactor either before or after prior sulphurization. The prior sulphurization was carried out in the gas phase with a H₂S/H₂ mixture in which the quantity of H₂S was 15% by volume, at a temperature of 350° C. for 2 h.

The feed contained 20% of toluene, 5.88% of dimethyldisulphide (CH₃—S—S—CH₃, sulphurization agent) and 74.12% of cyclohexane (as solvent). This liquid feed was mixed with a flow of hydrogen. The ratio of the hydrogen flow rate to the liquid feed flow rate was 450 L H₂ (at 0° C. and atmospheric pressure) per L of liquid feed (based on the density at 15° C.).

The reactor was pressurized to 60 bar (6 MPa). The feed flow rate corresponded to an hourly space velocity (HSV) of 4 h⁻¹. The temperature was slowly increased to 350° C. (ramp-up of 2° C./min). After 2 h at 350° C., the HSV was reduced to 2 h⁻¹.

The catalytic activity was evaluated after a stabilization period of at least 4 h.

Effluent samples were analysed by gas phase chromatography. The disappearance of the toluene was evaluated.

This test was repeated at a temperature of 370° C. and 390° C.

The catalytic performances are summarized in the table below:

| | Formulation | Toluene | Toluene | Toluene |
|---|---|---|---|---|
| | (% by weight | conversion, | conversion, | conversion, |
| | CoO, MoO₃, P₂O₅, | % | % | % |
| Catalyst | % dop) | 350° C. | 350° C. | 390° C. |
| Cdop1 | CoMoP/Pdop @ | 43 | 60 | 65 |
| | Al₂O₃-1 | | | |
| | (4.4/20/0.8/5.2) | | | |
| C1 | CoMoP/Al₂O₃-1 | 32 | 45 | 50 |
| | (4.4/20/0.8/-) | | | |
| Cdop2 | CoMoP/Pdop @ | 21 | 33 | 41 |
| | Al₂O₃-1 | | | |
| | (2.3/10/0.4/5.2) | | | |
| C2 | CoMoP/Al₂O₃-1 | 18 | 25 | 31 |
| | (2.3/10/0.4/-) | | | |
| Cdop3 | CoMoP/Pdop @ | 53 | 69 | 74 |
| | Al₂O₃-2 | | | |
| | (4.5/20/0.8/11.2) | | | |
| C3 | CoMoP/Al₂O₃-2 | 41 | 54 | 61 |
| | (4.5/20/0.8/-) | | | |
| Cdop4 | CoMoP/Pdop @ SiO₂ | 22 | 33 | 42 |
| | (4.5/20/0.8/11) | | | |
| C4 | CoMoP/SiO₂ | 19 | 25 | 30 |
| | (4.5/20/0.8/-) | | | |
| Cdop5 | CoMoP/Pdop @ | 45 | 61 | 68 |
| | Al₂O₃-3 | | | |
| | by Comixing support | | | |
| | (4.6/20/0.8/8.1) | | | |
| Cdop6 | CoMoP/Pdop @ | 21 | 31 | 40 |
| | Al₂O₃-1 | | | |
| | by Co-impregnation | | | |
| | (2.2/10/0.4/5.2) | | | |
| Cdop7 | CoMoP/Pdop @ | 24 | 33 | 43 |
| | Al₂O₃-3 | | | |
| | Comixing complex | | | |
| | (4.6/20/-/38) | | | |
| Cdop8 | (Pdop/CoMoP/Al₂O₃) | 35 | 48 | 51 |
| | Post-additive addition | | | |
| | (4.4/20/0.8/5.2) | | | |

It can be seen that all of the catalysts in accordance with the invention had improved conversions compared with their homologues without dopamine, and thus had an improved activity.

### Example 10: Catalytic Test: Desulphurization of 3-methylthiophene in Competition with the Hydrogenation of 2,3-dimethyl-2-butene

This catalytic test was aimed at evaluating the activity and selectivity of a hydrotreatment catalyst for the HDS of a cracked gasoline.

The mass of catalysts corresponding to a bed volume of 0.30 cm³ was charged into a fixed flushed bed reactor either before or after prior sulphurization. The sulphurization step was carried out in the gas phase with a H₂S/H₂ mixture in which the quantity of H₂S was 15% by volume, at a temperature of 350° C. for 2 h.

The catalyst charged into the reactor was initially sulphurized with a feed containing 4% of DMDS and 96% by weight of n-heptane. The liquid feed was mixed with a flow of H₂ (300 L H₂ per L of liquid feed). The pressure was adjusted to 15 bar (1.5 MPa). The temperature was increased at a ramp-up of 2° C./min to 350° C. and maintained at 350° C. for 2 h.

Next, the temperature was dropped to 190° C. and the sulphurization feed was replaced with the test feed.

The test feed contained 10% by weight of 2,3-dimethylbut-2-ene, 0.30% by weight of 3-methylthiophene and 89.7% by weight of n-heptane (as a solvent).

The flow rate of liquid feed corresponded to an hourly space velocity (HSV) of 6 h⁻¹.

The temperature was increased from 190° C. to 220° C. in intervals of 10° C.

At each temperature, the disappearance of 3-methylthiophene as well as the formation of hydrogenation products of 2,3-dimethylbut-2-ene were measured by analysis of the effluents using gas phase chromatography.

The conversion of 3-methylthiophene was calculated via the disappearance of the 3-methylthiophene.

The selectivity of the catalyst was evaluated via the appearance of the reaction products.

Selectivity=k(HDS)/k(HYD)

The first order constant k (g_{feed} g_{MoO3}⁻¹ h⁻¹) was calculated using the following equation:

k (HDS or HYD)=WHSV*ln (1/(1−x)

in which WHSV=(flow rate (feed)*ρ(feed))/m(MoO₃),
x=conversion of 3-methyl-thiophene or hydrogenated products of 2,3-dimethylbut-2-ene.

The catalytic performances at 200° C. are expressed in the following table:

| | | 3-methyl- | Hydrogenated | |
|---|---|---|---|---|
| | | thiophene | products of 2,3- | |
| | | conversion, | dimethyl- | |
| Catalyst | | % | but-2-ene % | Selectivity |
| Cdop1 | CoMoP/Pdop @ | 45.6 | 4.3 | 14 |
| | Al₂O₃-1 | | | |
| C1 | CoMoP/Al₂O₃-1 | 61.0 | 8.3 | 11 |
| Cdop3 | CoMoP/Pdop @ | 72.2 | 10.1 | 12.0 |
| | Al₂O₃-2 | | | |
| C3 | CoMoP/Al₂O₃-2 | 73.7 | 21.4 | 5.5 |
| Cdop4 | CoMoP/Pdop @ | 44.2 | 3.0 | 19 |
| | SiO₂ | | | |
| C4 | CoMoP/SiO₂ | 29.2 | 1.5 | 23 |

It can be seen that all of the catalysts in accordance with the invention on the Al₂O₃ support had improved selectivities compared with their homologues without dopamine. The catalyst on the SiO₂ support had an improved activity compared with its homologue without dopamine.

### Example 11: Evaluation of Catalysts C1 and C2 (Comparative) and C1dop and C2dop Using Gas Oil HDS

The catalysts C1, C2, C1dop and C2dop were tested using gas oil HDS. The gas oil feed used was a mixture of straight-run gas oil and Light Cycle Oil (LCO). The quantity of sulphur was 0.6815% by weight. The quantity of nitrogen was 488 mg/dm³. The density at 15° C. was 0.8795 g/cm³.

The test was carried out in an isothermal fixed bed flushed reactor. After sulphurization in situ at 350° C. in the unit pressurized with test gas oil to which 2% by weight of dimethyldisulphide had been added, the hydrodesulphurization test was carried out under the following operating conditions: a total pressure of 4 MPa, a catalyst volume of 0.48 cm³, a temperature of 330° C. to 340° C., a hydrogen flow rate of 2.56 cm³/min and a feed flow rate of 0.48 cm³/h.

The table below indicates the S contents (in ppm, i.e. in μg S/g gas oil) in the effluent from the reactor. These contents were measured after a 10 day stabilization period for the first temperature and 5 days for the following two temperatures. It can clearly be seen that the catalyst prepared with dopamine has a better desulphurizing activity than its analogue prepared without dopamine.

| | | S content (ppm) | | |
|---|---|---|---|---|
| Catalyst | | 330° C. | 335° C. | 340° C. |
| Cdop1 | CoMoP/Pdop @ Al₂O₃-1 | 496 | 363 | 274 |
| C1 | CoMoP/Al₂O₃-1 | 595 | 463 | 363 |
| Cdop2 | CoMoP/Pdop @ Al₂O₃-2 | 167 | 119 | 83 |
| C2 | CoMoP/Al₂O₃-2 | 258 | 188 | 138 |

1. A catalyst comprising a support based on alumina or silica or silica-alumina, at least one element selected from group VIII and/or group VIB, and at least one catecholamine.

2. The catalyst as claimed in claim 1, in which the catecholamine is selected from dopamine, noradrenaline, adrenaline and isoprenaline, alone or as a mixture.

3. The catalyst as claimed in claim 1, in which the content of the element from group VIB is in the range 5% to 40% by weight, expressed as the oxide of the metal from group VIB with respect to the total weight of catalyst, and the content of the element from group VIII is in the range 1% to 10% by weight, expressed as the oxide of the metal from group VIII with respect to the total weight of catalyst.

4. The catalyst as claimed in claim 1, further containing phosphorus, the quantity of phosphorus being in the range 0.01% to 20% by weight, expressed as P₂O₅ with respect to the total weight of catalyst, and the ratio of phosphorus to the element from group VIE in the catalyst being greater than or equal to 0.01.

5. The catalyst as claimed in claim 1, in which the quantity of catecholamine is in the range 1% to 40% by weight with respect to the weight of the support.

6. The catalyst as claimed in claim 1, further containing an organic compound other than catecholamine, containing oxygen and/or nitrogen and/or sulphur.

7. The catalyst as claimed in claim 6, in which the organic compound is selected from a compound comprising one or more chemical functions selected from a carboxyl, alcohol, thiol, thioether, sulphone, sulphoxide, ether, aldehyde, ketone, ester, carbonate, amine, nitrile, imide, oxime, urea and amide function.

8. The catalyst as claimed in claim 1, characterized in that it is at least partially sulphurized.

9. A process for the preparation of a catalyst as claimed in claim 1, comprising the following steps:
a) bringing at least one component of an element from group VIE and/or at least one component of an element from group VIII, at least one catecholamine and optionally phosphorus, into contact with a support based on alumina or silica or silica-alumina, in a manner such as to obtain a catalyst precursor,
b) drying said catalyst precursor obtained from step a) at a temperature of less than 200° C., without subsequently calcining it.

10. The process as claimed in claim 9, in which step a) comprises the following steps:
a1) preparing a support comprising a catecholamine,
a2) impregnating the support obtained in step a1) with an impregnation solution comprising at least one element from group VIE and/or at least one element from group VIII and optionally phosphorus in a manner such as to obtain a catalyst precursor.

11. The process as claimed in claim 10, in which in step a1), the support comprising a catecholamine is prepared by introducing a catecholamine at any time during the preparation of the support, and preferably during shaping of the support, or by impregnation onto a support which has already been shaped.

12. The process as claimed in claim 9, in which step a) comprises the following steps:
a1′) bringing a solution containing at least one element from group VIB and/or at least one element from group VIII, at least one catecholamine and optionally phosphorus into contact, by co-impregnation, with a support based on alumina or silica or silica-alumina in a manner such as to obtain a catalyst precursor.

13. The process as claimed in claim 9, in which step a) comprises the following steps:
a1″) impregnating a support based on alumina or silica or silica-alumina with at least one solution containing at least one element from group VIE and/or at least one element from group VIII and optionally phosphorus in order to obtain an impregnated support,
a2″) drying the impregnated support obtained in step a1″) at a temperature of less than 200° C. in order to obtain a dried impregnated support, and optionally calcining the dried impregnated support in order to obtain a calcined impregnated support,
a3″) impregnating the dried and optionally calcined impregnated support obtained in step a2″) with an impregnation solution comprising a catecholamine in a manner such as to obtain a catalyst precursor.

14. The process as claimed in claim 9, in which the catecholamine is dopamine.

15. A process for the hydrotreatment and/or hydrocracking of hydrocarbon cuts, comprising subjecting a hydrocarbon cut to hydrotreatment and/or hydrocracking in the presence of a catalyst according to claim 1.

16. A process for the hydrotreatment and/or hydrocracking of a hydrocarbon cut, comprising subjecting a hydrocarbon cut to hydrotreatment and/or hydrocracking in the presence of a catalyst prepared by a process of claim 9

17. A process for the hydrotreatment and/or hydrocracking of a hydrocarbon cut, comprising preparing a catalyst by
a) bringing at least one component of an element from group VIB and/or at least one component of an element from group VIII, at least one catecholamine and optionally phosphorus, into contact with a support based on alumina or silica or silica-alumina, in a manner such as to obtain a catalyst precursor,
b) drying said catalyst precursor obtained from step a) at a temperature of less than 200° C., without subsequently calcining it and subjecting said cut to hydrotreatment and/or hydrocracking in the presence of said catalyst.
